(19) United States
(12) Patent Application Publication
(10) Pub. No.: US 2014/0000606 A1
(43) Pub. Date: Jan. 2, 2014

(54) METHODS AND SYSTEMS FOR MIMICKING FLUCTUATIONS IN DELIVERED FLOW AND/OR PRESSURE DURING VENTILATION

(71) Applicants: Peter R. Doyle, Vista, CA (US); Gardner Kimm, Carlsbad, CA (US)

(72) Inventors: Peter R. Doyle, Vista, CA (US); Gardner Kimm, Carlsbad, CA (US)

(73) Assignee: Nellcor Puritan Bennett LLC, Boulder, CO (US)

(21) Appl. No.: 13/539,716

(22) Filed: Jul. 2, 2012

(51) Int. Cl. A61M 16/00

(52) U.S. Cl. 128/204.21

(57) ABSTRACT

This disclosure describes systems and methods for a ventilator-derived CPAP system that mimics the flow and/or pressure oscillations or fluctuations of the B-CPAP system creating a breath type referred to herein as a mimicked-bubble-CPAP (M-CPAP) mode. Further, the disclosure describes systems and methods for delivery of other breath types with flow and/or pressure oscillations or fluctuations that mimic the oscillation observed during ventilation with the B-CPAP system, referred to herein as adjusted breath types.

Medical ventilator systems have long been used to provide ventilatory and supplemental oxygen support to patients. These ventilators typically comprise a source of pressurized oxygen which is fluidly connected to the patient through a conduit or tubing. As each patient may require a different ventilation strategy, modern ventilators can be customized for the particular needs of an individual patient. For example, ventilators may control the amount of pressure or flow delivered to a patient during ventilation.

Mimicking Fluctuations in Delivered Flow and/or Pressure During Ventilation

This disclosure describes systems and methods for a ventilator-derived CPAP system that mimics the flow and/or pressure oscillations or fluctuations of the B-CPAP system creating a mode referred to herein as a mimicked-bubble-CPAP (M-CPAP) mode. Further, the disclosure describes systems and methods for delivery of breath types with flow and/or pressure oscillations or fluctuations that mimic the oscillations observed during ventilation with the B-CPAP system, referred to herein as adjusted breath types.

In part, this disclosure describes a method for ventilating a patient with a ventilator. The method includes:

a) receiving a user selected Continuous Positive Airway Pressure (CPAP) level;

b) generating a control signal for controlling breathing gas delivery and an exhalation valve to implement the user selected CPAP level;

c) generating a normalized bubble signal;

d) adding the control signal and the normalized bubble signal to form a mimic signal; and

e) delivering the user selected CPAP level to the patient with fluctuations in at least one of flow and pressure based on the mimic signal.

In part, this disclosure describes a method for ventilating a patient with a ventilator. The method includes:

a) receiving a breath type;

b) generating a control signal for controlling breathing gas delivery and an exhalation valve to implement the breath type;

c) generating a normalized bubble signal;

d) adding the control signal and the normalized bubble signal to form a mimic signal; and

e) delivering the breath type to the patient with fluctuations in at least one of flow and pressure based on the mimic signal.

Additionally, this disclosure describes a ventilator system that includes: a pressure generating system, a ventilation tubing system, and a bubble mimic module. The pressure generating system is adapted to generate a flow of breathing gas. The pressure generating system includes an exhalation valve. The exhalation valve controls release of respiratory gases from a patient to the atmosphere. The ventilation tubing system includes a patient interface for connecting the pressure generating system to the patient. The bubble mimic module generates a normalized bubble signal and adds the normalized bubble signal to a control signal for a breath type to form a mimic signal. The pressure generating system delivers the breath type to the patient with fluctuations in at least one of flow and pressure based on the mimic signal from the bubble mimic module.

The disclosure also describes a ventilator system including means for receiving a breath type; means for generating a control signal for operating at least one of an exhalation valve and a pressure generating system to provide the breath type; means for generating a normalized bubble signal for operating the at least one of the exhalation valve and the pressure generating system; means for adding the control signal and the normalized bubble signal to form a mimic signal; means for sending the mimic signal to the at least one of the exhalation valve and the pressure generating system; and means for delivering the breath type to a patient with fluctuations in at least one of flow and pressure based on the mimic signal.

The disclosure additionally describes a computer-readable medium having computer-executable instructions for performing a method for ventilating a patient with a ventilator. The method includes:

a) receiving a breath type;

b) repeatedly generating a control signal for operating at least one of an exhalation valve and a pressure generating system to provide the breath type;

c) generating a normalized bubble signal for operating the at least one of the exhalation valve and the pressure generating system;

d) repeatedly adding the control signal and the normalized bubble signal to form a mimic signal;

e) repeatedly sending the mimic signal to the at least one of the exhalation valve and the pressure generating system; and

f) repeatedly delivering the breath type to the patient with fluctuations in at least one of flow and pressure based on the mimic signal

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The following drawing figures, which form a part of this application, are illustrative of embodiments of systems and methods described below and are not meant to limit the scope of the invention in any manner, which scope shall be based on the claims.

FIG. 1A illustrates an embodiment of a ventilator.

FIG. 1B illustrates an embodiment of a signal system on a ventilator.

FIG. 2 illustrates an embodiment of a method for delivering a M-CPAP mode during ventilation of a patient on a ventilator.

FIG. 3 illustrates an embodiment of a method for delivering an adjusted breath type during ventilation of a patient on a ventilator.

FIG. 4 illustrates an embodiment of a graph of delivered pressure by a ventilator during a CPAP mode and delivered pressure by the ventilator during M-CPAP mode.

FIG. 5 illustrates an embodiment of a graph of delivered flow by a ventilator during a CPAP mode and delivered flow by the ventilator during M-CPAP mode.

FIG. 6 illustrates an embodiment of a graph of recorded delivered pressure fluctuations during ventilation of a patient by a ventilator utilizing a B-CPAP system.

FIG. 7 illustrates an embodiment of a graph of delivered pressure by a ventilator during a VC breath type and of delivered pressure by the ventilator during an adjusted VC breath type.

FIG. 8 illustrates an embodiment of a graph of delivered flow by a ventilator during a VC breath type and of delivered flow by the ventilator during an adjusted VC breath type.

FIG. 9 illustrates an embodiment of a graph of normalized pressure fluctuations based on pressure fluctuation observed in the graph illustrated in FIG. 6.

FIG. 10 illustrates an embodiment of a graph of delivered pressure by a ventilator during a PAV breath type and of delivered pressure by the ventilator during an adjusted PAY breath type.

FIG. 11 illustrates an embodiment of a graph of delivered flow by a ventilator during a PAV breath type and of delivered flow by the ventilator during an adjusted PAV breath type.

FIG. 12 illustrates an embodiment of a graph of recorded delivered flow fluctuations during ventilation of a patient by a ventilator utilizing a B-CPAP system.

FIG. 13 illustrates an embodiment of a method for delivering a M-CPAP mode during ventilation of a patient on a ventilator.

### DETAILED DESCRIPTION

Although the techniques introduced above and discussed in detail below may be implemented for a variety of medical devices, the present disclosure will discuss the implementation of these techniques in the context of a medical ventilator for use in providing ventilation support to a human patient. A person of skill in the art will understand that the technology described in the context of a medical ventilator for human patients could be adapted for use with other systems such as ventilators for non-human patients and general gas transport systems.

Medical ventilators are used to provide a breathing gas to a patient who may otherwise be unable to breathe sufficiently. In modern medical facilities, pressurized air and oxygen sources are often available from wall outlets. Accordingly, ventilators may provide pressure regulating valves (or regulators) connected to centralized sources of pressurized air and pressurized oxygen. The regulating valves function to regulate the flow of air and oxygen so that respiratory gas with a desired concentration of oxygen is supplied to the patient at a desired pressure and rate. Ventilators capable of operating independently of external sources of pressurized air and oxygen are also available.

As each patient may require a different ventilation strategy, modern ventilators can be customized for the particular needs of an individual patient. For example, several different ventilator modes or settings have been created to provide better ventilation for patients in various different scenarios, such as pressure ventilation, continuous positive airway pressure (CPAP), and Positive End Exhalation Pressure (PEEP).

The CPAP mode involves delivering breathing gases to a spontaneously-breathing patient by targeting a constant pressure at a clinician-selected level at the patient's airway. When delivering the CPAP mode, an additional pressure above the CPAP level (e.g., 1.5 cmH₂O) may be provided. CPAP may be applied invasively (via endotracheal tube) or non-invasively (via a nasal mask interface) to prevent the collapse of alveoli. CPAP may be beneficial for premature neonatal patients or patients with respiratory diseases, such as sleep apnea.

An early method for applying CPAP during ventilation involved the continuous flow of gas through a water column to provide the desired level of positive pressure and is referred to herein as a “bubble CPAP system” or “B-CPAP system”. The amount of pressure delivered by a B-CPAP system is dependent upon the amount of water in the water column. Newer systems for applying CPAP during ventilation are performed by adjusting the flow rate and/or the exhalation valve to control the amount of pressure delivered to the patient during ventilation and is referred to herein as a “ventilator-derived CPAP system” or “V-CPAP system”. Accordingly, the amount of pressure delivered to the patient during ventilation on a ventilator with a V-CPAP system is adjusted by controlling gas delivery and/or the exhalation valve.

A bubble CPAP system typically consists of a flow metering valve, a flow meter and a container of water. This simple system has no monitoring capabilities. A water column could be integrated into a modern ventilator, but then other issues arise. For example, having to establish and maintain a water level make utilizing a water column impractical. Also, the pressure and flow oscillations will cause problems for the ventilator as it uses the pressure and flow signals for detection of triggering and cycling events, calculation of exhaled volumes, alarm activation, etc. Accordingly, the use of a water column in the bubble CPAP system makes monitoring of pressure and/or flow difficult and/or inefficient (i.e., exhalation flow, delivered flow, leakage, etc.), which in turn prevents alarm determinations based on these monitored parameters. Additionally, the water level in the B-CPAP system has to be kept constant to maintain the desired amount of pressure, which may slowly change due to evaporation. Further, the water and column have to be kept clean during use of the B-CPAP system. Moreover, products utilizing a B-CPAP system typically do not provide for triggering of breaths due to the difficulties involved with trigger detection caused by the flow and pressure oscillations. Therefore, products utilizing a B-CPAP system typically had to be switched with other ventilator systems to provide triggered ventilation to a patient when necessary. Accordingly, most patients were transitioned from the B-CPAP system to the V-CPAP system.

However, a recent study has suggested that the B-CPAP system provide better gas exchange when compared to the newer V-CPAP systems.¹ This study found statistically significant changes in vital signs from baseline measurements, such as a statistically significant decrease in respiration rate (RR), pH, and PaO₂ with a corresponding statistically significant increase in PaCO₂, HCO₃ during use of the V-CPAP system during ventilation of a patient on a ventilator. However, no significant changes from the baseline measurements for these vital signs were observed during the use of a B-CPAP system during ventilation. Therefore, the patients ventilated with the B-CPAP system showed improved gas exchange compared to patients ventilated with the V-CPAP system. It is hypothesized that this observed improvement in gas exchange is the result of oscillations in the flow and/or pressure delivered by the bubble CPAP system resulting from bubbles moving through the water column. ¹Huang, W C et al., “Comparison Between Bubble CPAP and Ventilation-Derived CPAP in Rabbits,” Pediatr Neonatal. 2009 February; 50(1):39.

Accordingly, the systems and methods described herein provide for a ventilator-derived CPAP system that mimics the flow and/or pressure oscillations or fluctuations of the B-CPAP system creating a mode referred to herein as a mimicked-bubble-CPAP (M-CPAP) mode. The terms “oscillation” and “fluctuation” are intended to be interchangeable when utilized herein. Further, the systems and methods described herein further provide for the delivery of other breath types with flow and/or pressure oscillations or fluctuations that mimic the oscillation observed during ventilation with the B-CPAP system, which are referred to herein as adjusted breath types. The ventilator system mimics the flow and/or pressure oscillations or fluctuations of the B-CPAP system during an adjusted mode, such as M-CPAP, by controlling the gas delivery and/or the exhalation valve in the ventilator. The capabilities that are typically part of a modern ventilator allow for monitoring of flow and/or pressure. The monitoring of flow and/or pressure allow for leakage monitoring, alarming based on monitored flow and/or pressure, and triggered breath types in ventilators with M-CPAP systems. Also, since the ventilator controls the oscillatory signal injected into the ventilator tubing system, the ventilator can ‘filter’ these oscillations back out in order to improve the ability for monitoring the flow and/or pressure signals with M-CPAP systems.

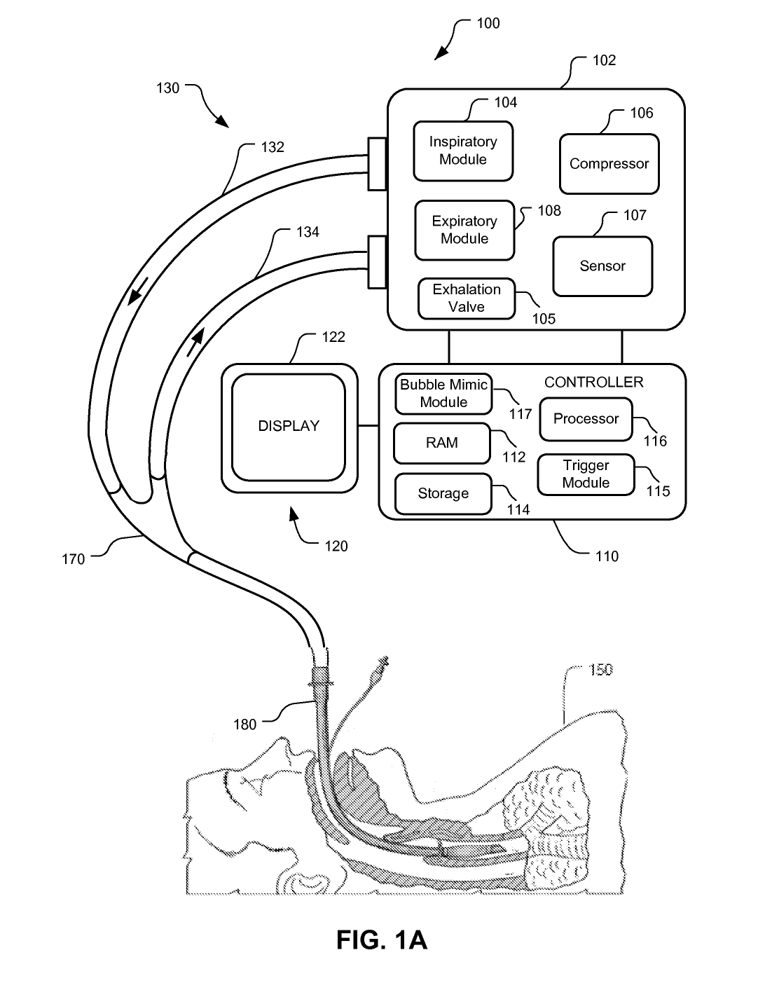

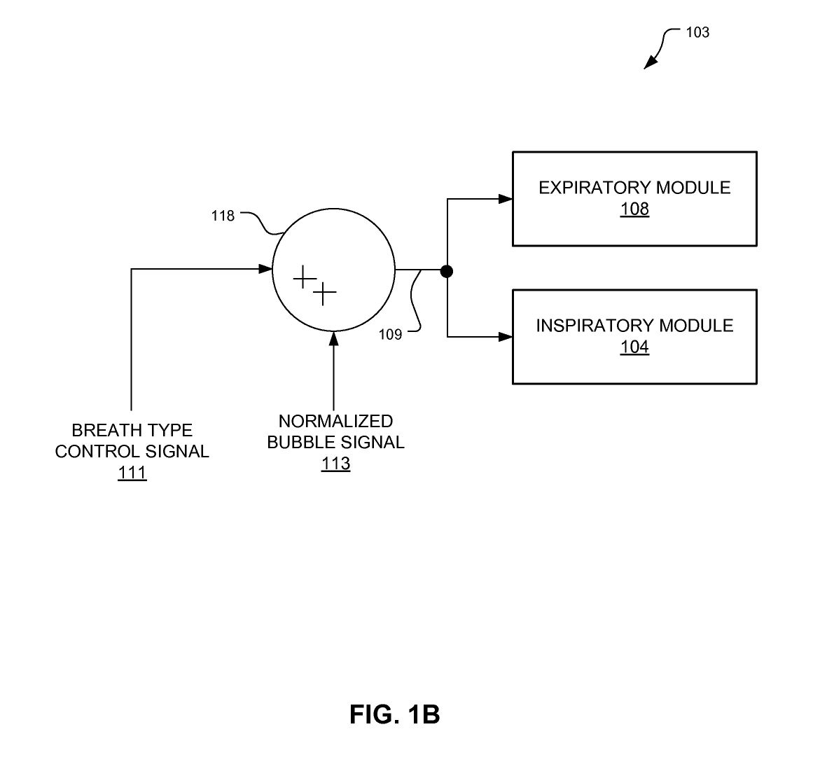

FIG. 1A illustrates an embodiment of an exemplary ventilator 100. The exemplary ventilator 100 illustrated in FIG. 1A is connected to a human patient 150. Ventilator 100 includes a pneumatic system 102 (also referred to as a pressure generating system 102) for circulating breathing gases to and from patient 150 via the ventilation tubing system 130, which couples the patient 150 to the pneumatic system 102 via an invasive (e.g., endotracheal tube, as shown) or a non-invasive (e.g., nasal mask) patient interface 180.

Ventilation tubing system 130 (or patient circuit 130) may be a two-limb (shown) or a one-limb circuit for carrying gases to and from the patient 150. In a two-limb embodiment, a fitting, typically referred to as a “wye-fitting” 170, may be provided to couple the patient interface 180 (shown as an endotracheal tube in FIG. 1A and as a nasal mask in FIG. 1B) to an inspiratory limb 132 and an expiratory limb 134 of the ventilation tubing system 130.

Pneumatic system 102 may be configured in a variety of ways. In the present example, pneumatic system 102 includes an expiratory module 108 coupled with the expiratory limb 134 and an inspiratory module 104 coupled with the inspiratory limb 132. Compressor 106, accumulator and/or other source(s) of pressurized gases (e.g., air, oxygen, and/or helium) is coupled with inspiratory module 104 and the expiratory module 108 to provide a gas source for ventilatory support via inspiratory limb 132.

The inspiratory module 104 is configured to deliver gases to the patient 150 through the inspiratory limb 132 according to prescribed ventilatory settings. In some embodiments, the inspiratory module 104 is associated with and/or controls an inspiratory valve for controlling gas delivery to the patient 150 through the inspiratory limb 132. In some embodiments, inspiratory module 104 is configured to provide ventilation according to various ventilator modes and breath types, such as a mandatory mode, a spontaneous mode, a CPAP mode, a pressure assist breath type, a proportional assist breath type or any other known mode and/or breath type. In some embodiments, the breath types delivered are adjusted breath types.

In some embodiments, the inspiratory module 104 delivers gases to the patient according to volume-control (VC). The VC breath type allows a clinician to set a respiratory rate and to select a volume to be administered to a patient during a mandatory breath. When using VC, a clinician sets a desired tidal volume, flow wave form shape, and an inspiratory flow rate or inspiratory time. These variables determine how much volume of gas is delivered to the patient and the duration of inspiration during each mandatory breath inspiratory phase. The mandatory breaths are administered according to the set respiratory rate.

For VC, when the delivered volume is equal to the prescribed tidal volume, the ventilator may initiate exhalation. Exhalation lasts from the time at which prescribed volume is reached until the start of the next ventilator mandated inspiration. This exhalation time is determined by the respiratory rate set by the clinician and any participation above the set rate by the patient. Upon the end of exhalation, another VC mandatory breath is given to the patient.

During VC, delivered volume and flow waveforms may remain constant and may not be affected by variations in lung or airway characteristics. Alternatively, pressure readings may fluctuate based on lung or airway characteristics. According to some embodiments, the ventilator may control the inspiratory flow and then derive volume based on the inspiratory flow and elapsed time.

In further embodiments, the inspiratory module 104 is configured to deliver gases to the patient using a volume-targeted-pressure-control (VC+) breath type. The VC+ breath type is a combination of volume and pressure control breath types that may be delivered to a patient as a mandatory breath. In particular, VC+ may provide the benefits associated with setting a target tidal volume, while also allowing for variable flow. Variable flow may be helpful in meeting inspiratory flow demands for actively breathing patients.

As may be appreciated, when resistance increases it becomes more difficult to pass gases into and out of the lungs, decreasing flow. For example, when a patient is intubated, i.e., having either an endotracheal or a tracheostomy tube in place, resistance may be increased as a result of the smaller diameter of the tube over a patient's natural airway. In addition, increased resistance may be observed in patients with obstructive disorders, such as COPD, asthma, etc. Higher resistance may necessitate, inter alia, a higher inspiratory time setting for delivering a prescribed pressure or volume of gases, a lower respiratory rate resulting in a higher expiratory time for complete exhalation of gases.

Unlike VC, when the set inspiratory time is reached, the ventilator may initiate exhalation. Exhalation lasts from the end of inspiration until the beginning of the next inspiration. For a non-triggering patient, the expiratory time (T_{E}) is based on the respiratory rate set by the clinician. Upon the end of exhalation, another VC+ mandatory breath is given to the patient.

By controlling target tidal volume and allowing for variable flow, VC+ allows a clinician to maintain the volume while allowing the flow and pressure targets to fluctuate.

In some embodiments, the inspiratory module 104 is configured to deliver gases to the patient according to volume-support (VS) breath type. The VS breath type is utilized in the present disclosure as a spontaneous breath. VS is generally used with a triggering (spontaneously breathing) patient when the patient is ready to be weaned from a ventilator or when the patient cannot do all of the work of breathing on his or her own. When the ventilator senses patient inspiratory effort, the ventilator delivers a set tidal volume during inspiration. The tidal volume may be set and adjusted by the clinician. The patient controls the rate, inspiratory flow, and has some control over the inspiratory time. The ventilator then adjusts the pressure over several breaths to achieve the set tidal volume. When the machine senses a decrease in flow, or inspiration time reaches a predetermined limit, the ventilator determines that inspiration is ending.

In additional embodiments, the inspiratory module 104 is configured to deliver gases to the patient according to the pressure-control (PC) breath type. PC allows a clinician to select a pressure to be administered to a patient during a mandatory breath. When using the PC breath type, a clinician sets a desired pressure, inspiratory time, and respiratory rate for a patient. These variables determine the pressure of the gas delivered to the patient during each mandatory breath inspiration. The mandatory breaths are administered according to the set respiratory rate.

For the PC breath type, when the inspiratory time is equal to the prescribed inspiratory time, the ventilator may initiate exhalation. Exhalation lasts from the end of inspiration until the next inspiration. Upon the end of exhalation, another PC mandatory breath is given to the patient.

During PC breaths, the ventilator may maintain the same pressure waveform at the mouth, regardless of variations in lung or airway characteristics, e.g., respiratory compliance and/or respiratory resistance. However, the volume and flow waveforms may fluctuate based on lung and airway characteristics.

In some embodiments, PC may also be delivered for triggering patients. When PC is delivered with triggering, the breath period (i.e. time between breaths) is a function of the respiratory rate of the patient. The ventilator will trigger the inhalation based upon the respiratory rate setting or the patient's trigger effort, but cycling to exhalation will be based upon elapsed inspiratory time. The inspiratory time is set by the clinician. The inspiratory flow is delivered based upon the pressure setting and patient physiology. Should the patient create an expiratory effort in the middle of the mandatory inspiratory phase, the ventilator will respond by reducing flow. If no patient effort is detected, the ventilator will deliver another mandatory breath at the predetermined respiratory rate.

In further embodiments, the inspiratory module 104 is configured to deliver gases to the patient according to a pressure-support (PS) breath type. PS is a form of spontaneous ventilation and is utilized in the present disclosure during a spontaneous breath. PS is a patient triggered breath and is typically used when a patient is ready to be weaned from a ventilator or for when patients are breathing spontaneously but cannot do all the work of breathing on their own. When the ventilator senses patient inspiratory effort, the ventilator provides a constant pressure during inspiration. The pressure may be set and adjusted by the clinician. The patient controls the rate, inspiratory flow, and to an extent, the inspiratory time. The ventilator delivers the set pressure and allows the flow to vary. When the machine senses a decrease in flow, or determines that inspiratory time has reached a predetermined limit, the ventilator determines that inspiration is ending.

The breath types described in detail above are exemplary only and are not meant to limit the disclosure. Any known breath types may be delivered to the patient by the ventilator 100 and adjusted by the addition of the normalized bubble signal 113 by the ventilator 100 as discussed below and delivered to the patient 150.

Because the ventilator 100 utilizes a V-CPAP system, the inspiratory module 104 may switch between a mandatory mode and a spontaneous mode. For example, the ventilator may switch between a PC breath type, which is a mandatory breath type to a PAV breath type, which is a spontaneous breath type. The ventilator 100 may switch between the mandatory mode and a spontaneous mode based on monitored parameters or based on user selection.

According to embodiments, the ventilator generates a breath type control signal 111 for directing one or more components and/or modules of the ventilator to deliver breathing gases to a patient. The breath type control signal 111 is generated as the result of one or more algorithms that are designed to deliver a selected breath type based on one or more clinician-selected ventilator settings. Although referred to as a single control signal for delivering a particular breath type, it should be understood that breath type control signal 111 may comprise one or more commands directed to one or more components or modules of the ventilator to implement the selected breath type and other ventilator settings (e.g., inspiratory pressure, PEEP, frequency, O₂%, etc.). In some embodiments, the pressure generating system 102 determines the breath type control signal 111. In other embodiments, the breath type control signal 111 is determined by the controller 110. In still other embodiments, the breath type control signal 111 may be generated by any other suitable ventilator module or component. The breath type control signal 111 is implemented by the inspiratory module 104 during inspiration to control gas delivery according to the selected breath type, mode, and other ventilator settings (e.g., peak inspiratory pressure, flow acceleration percent, O₂%, etc.). According to embodiments, the inspiratory module 104 implements the breath type control signal 111 by commanding one or more inspiratory valves and/or the exhalation valve. For example, the exhalation valve may be controlled during inspiration to release gases when a pressure overshoot is detected. The breath type and/or mode may be determined by the ventilator 100 and/or may be selected by an operator/user. As discussed above, the ventilator 100 may determine the mode and/or breath type by monitoring ventilator parameters. Ventilator parameters as used herein refer to any parameter known by the ventilator, such as clinician input, ventilator settings, and monitored patient data.

The expiratory module 108 is configured to release gases from the patient's lungs during exhalation according to prescribed ventilatory settings. In some embodiments, expiratory module 108 is associated with and/or controls an exhalation valve 105 for releasing respiratory gases from the patient 150 to the atmosphere. The expiratory module 108 also controls the exhalation valve 105 based on the generated control signal 111 for the breath type and/or mode, as well as other ventilator settings (e.g., PEEP).

During the delivery of an adjusted breath type, such as VC, by the ventilator 100, the inspiratory module 104 controls gas delivery based on a mimic signal 109 by commanding the inspiratory valve(s) and/or the exhalation valve. As discussed above, controlling either or both of the gas delivery and/or the exhalation valve dictates the flow and/or pressure delivered to the patient. Moreover, when providing an adjusted breath type, the expiratory module 108 controls the release of exhalation gases from the patient by commanding the exhalation valve based on the mimic signal 109.

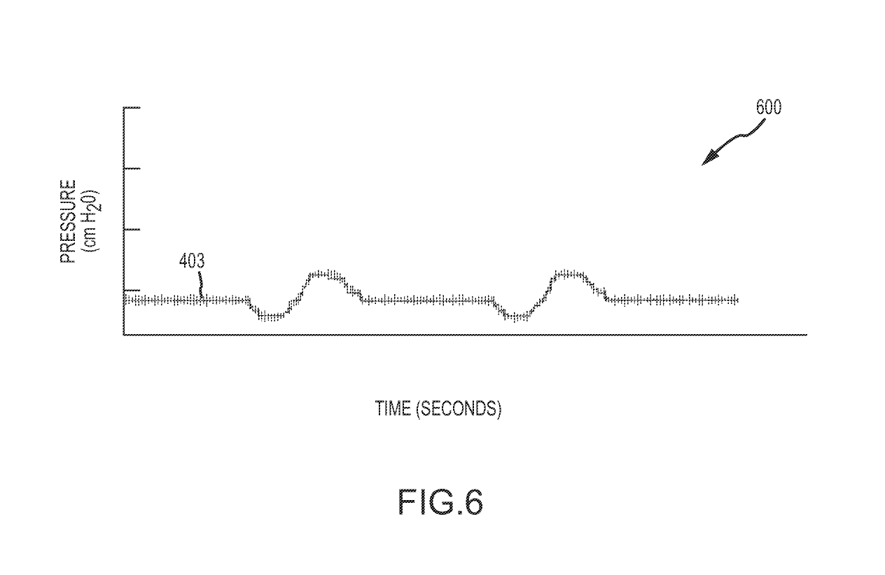

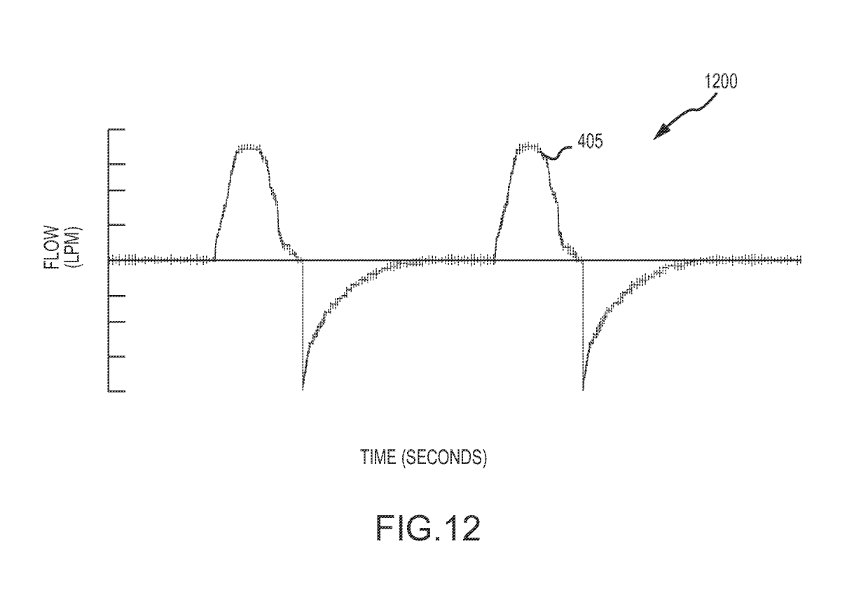

According to embodiments, the mimic signal 109 is received from a bubble mimic module 117. In some embodiments, the mimic signal 109 is based on recorded flow fluctuations and/or recorded pressure fluctuations during ventilation of a prior patient utilizing a B-CPAP system at a predetermined CPAP level for a set time period which is looped. The phrase “prior patient” or “different patient” as utilized herein includes ventilation with a B-CPAP system of an actual physical patient or refers to ventilation in a laboratory during ventilation of a simulated patient. FIG. 6 illustrates an embodiment of a graph 600 of delivered pressure fluctuations 403 recorded during ventilation of a prior patient by a ventilator utilizing a B-CPAP system. FIG. 12 illustrates an embodiment of a graph 1200 of delivered flow fluctuations 405 recorded during ventilation of a patient by a ventilator utilizing a B-CPAP system. In these embodiments, the mimic signal 109 is designed to mimic the pressure and/or flow fluctuations that were previously delivered to the prior patient for a set CPAP level for a set time period by controlling the expiratory valve and/or the gas delivery of the V-CPAP system. The mimic signal 109, in this embodiment, is looped in order to mimic fluctuation beyond the set time period of the recorded signal. In some embodiments, the ventilator 100 stores numerous different recorded flow fluctuations and/or recorded pressure fluctuations during ventilation of the same prior patient utilizing a B-CPAP system at any desired number of different CPAP level settings for the same set time period. The storing of numerous recorded fluctuations provides for generating numerous mimic signals 109, which allow the ventilator 100 to deliver substantially similar flow and/or pressure fluctuations as delivered by the ventilator with a B-CPAP system at the same set CPAP setting. The B-CPAP system variations that are replicated in the M-CPAP system may include CPAP level, bubble size, and/or bubble frequency.

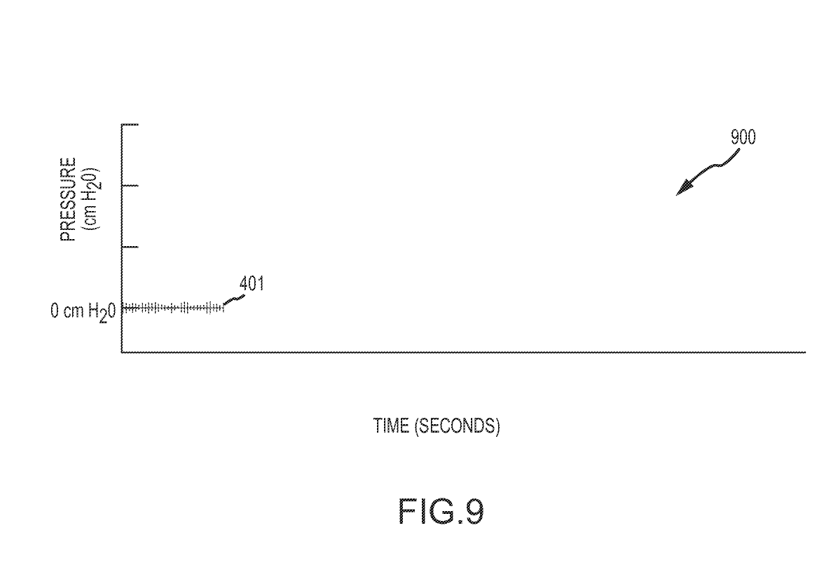

In other embodiments, the mimic signal 109 is determined by adding a normalized bubble signal 113 to a breath type control signal 111, which is illustrated in the signal system 103 shown in FIG. 1B. As discussed above, the normalized bubble signal 113 allows the amplitude of the oscillations to be changed in order to mimic a bubble CPAP system with different geometries and/or flows (e.g., larger bubbles). For example, a gain block may be added to change the amplitude of the normalized signal to simulate different size bubbles. In a similar manner a gain block could be added to the normalized signal to change the bubble frequency. In one embodiment, the normalized bubble signal 113 is normalization of a signal based on recorded flow fluctuations and/or recorded pressure fluctuations during ventilation of a prior patient utilizing a B-CPAP system at a predetermined CPAP level for a set time period which is looped. The normalization bubble signal determines how much the pressure and/or flow fluctuates from the intended delivered pressure and/or flow and then utilizes just these fluctuations above and below the intended flow and/or pressure delivery to determine the normalized signal. FIG. 9 illustrates an embodiment of a graph 900 of normalized pressure fluctuations based on pressure fluctuation observed in graph 600 of FIG. 6.

In an alternative embodiment, the normalized bubble signal is a normalized signal based on theoretical flow fluctuations and/or theoretical pressure fluctuations caused by mathematically estimated bubbles during ventilation of a patient utilizing a B-CPAP system at a predetermined CPAP level for a set time period which is randomized. Mathematical equations are known that mathematically estimate bubbles and the displacement of flow and/or pressure caused by these estimated bubbles in a predetermined water column. These equations may be utilized to estimate the fluctuation caused by bubbles in a water column of a B-CPAP system at different CPAP settings. These estimations may be utilized to fluctuate the flow and/or pressure delivered during the delivery of CPAP with a V-CPAP system. However, bubbles produced in the water column will appear randomly and create random fluctuations in the delivered pressure and flow during ventilation of a patient with a ventilator utilizing a B-CPAP system. Accordingly, the theoretically calculated pressure and/or flow oscillations may be randomized to account for the randomness of the B-CPAP delivery system. This randomization may be determined and then looped or continuously determined. Similar to the normalized bubble signal discussed above, the theoretically calculated fluctuations may be determined based on a set CPAP setting for a predetermined amount of time and then looped.

FIG. 1B illustrates an embodiment of a signal system 103 for generating or determining the mimic signal 109 and for delivering the mimic signal 109 to the expiratory module 108 for implementation during exhalation and the inspiratory module 104 for implementation during inhalation. The signal system 103 of FIG. 1B includes a signal adder 118 that adds a received breath type control signal 111 and a normalized bubble signal 113. The sum of the two signals creates the mimic signal 109. The mimic signal 109 is then sent by the signal adder 118 to the expiratory module 108 and/or to the inspiratory module 104. In some embodiments, the bubble mimic module 117 includes the signal adder 118. In some embodiments, the bubble mimic module 117 determines the breath type control signal 111. In alternative embodiments, the signal adder 118 is located in the controller 110 or the pressure generating system 102. In other embodiments, the controller 110 or the pressure generating system 102 determine the breath type control signal 111.

As described above, the breath type control signal 111 is an instruction signal for the components of the ventilator 100, such as the inspiratory module 104 and/or the expiration module 108, on how to deliver a breath type. The mimic signal 109 is an instruction signal for the components of the ventilator on how to deliver a breath type with recorded fluctuation in pressure and/or flow that were delivered during delivery of CPAP by a different ventilator to a prior patient with a B-CPAP system. As discussed above, the breath type may be determined by the ventilator 100 or selected by the operator.

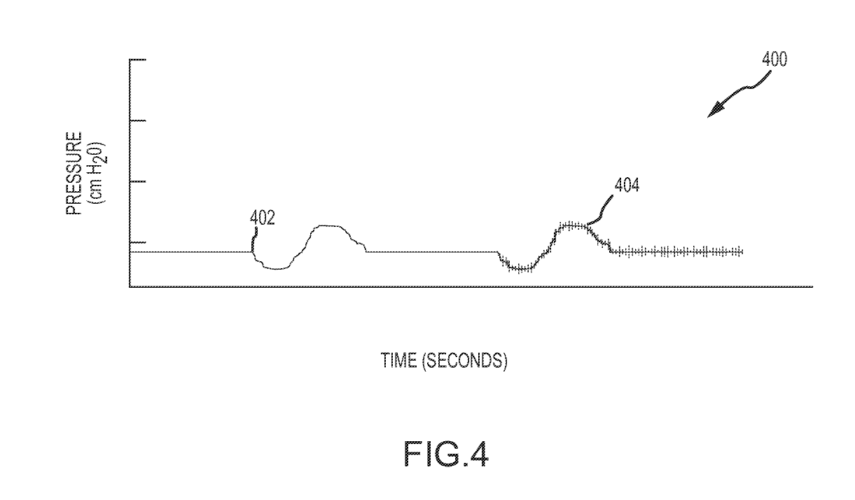

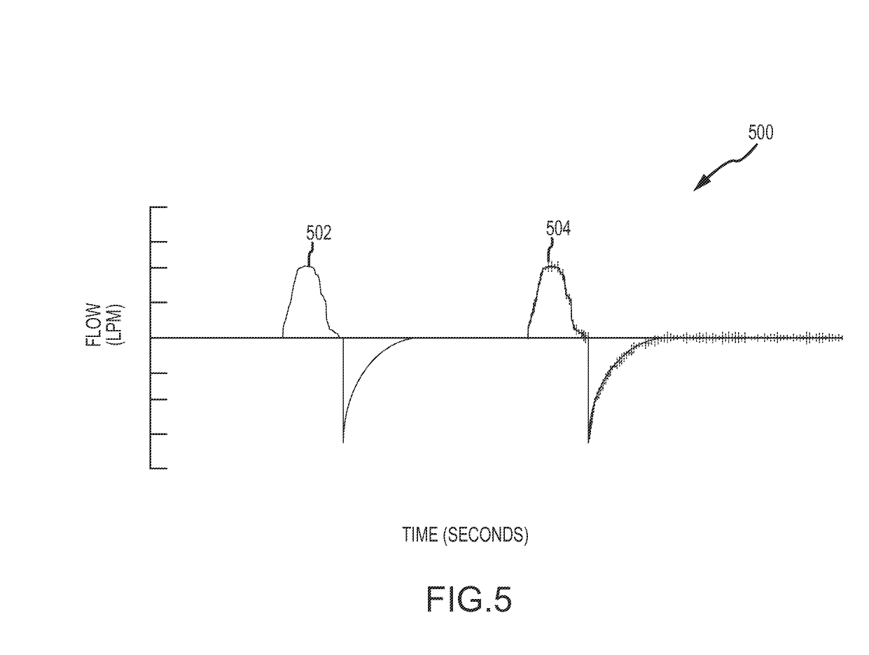

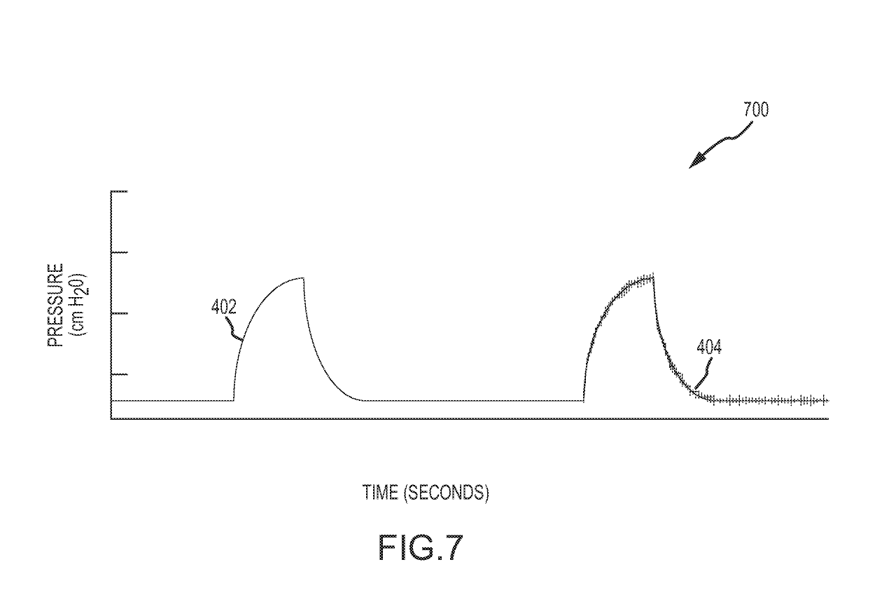

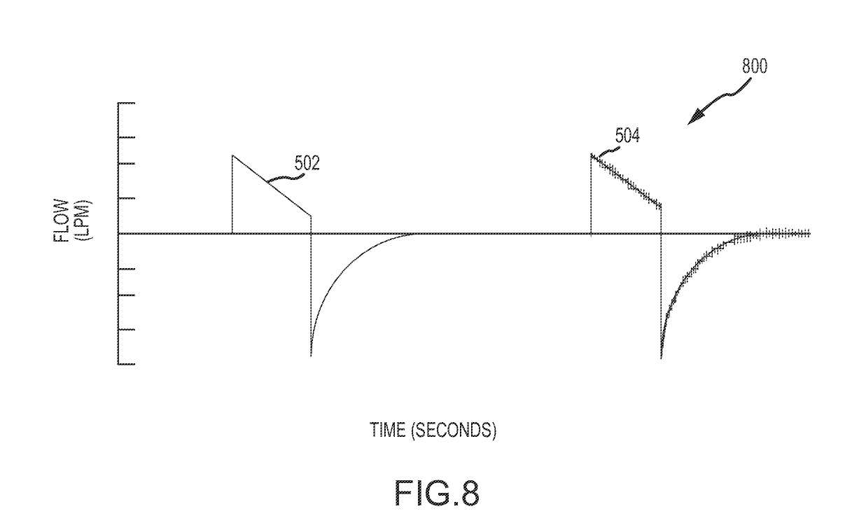

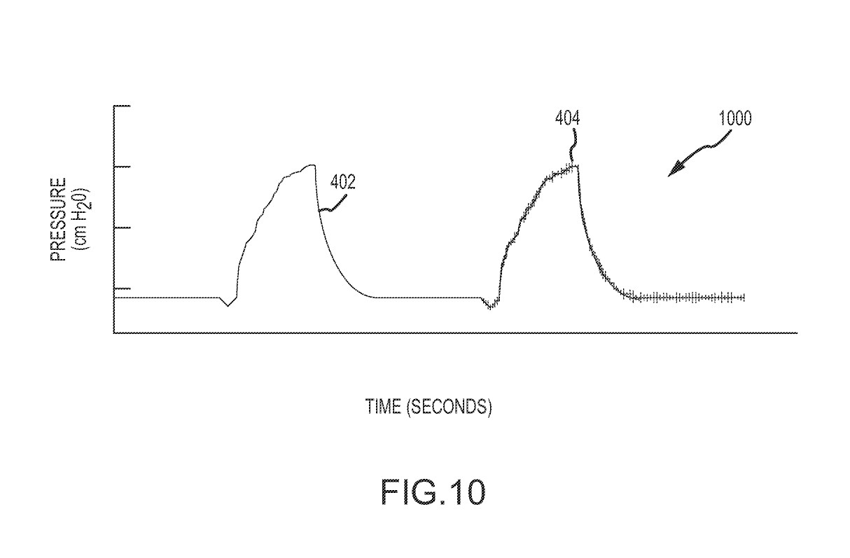

FIGS. 4, 7, and 10 illustrate embodiments of a graph showing the pressure delivered 402 during a traditional breath type and the pressure delivered 404 by the ventilator during an adjusted breath type (i.e. after the delivered pressure has been adjusted to include the normalized pressure fluctuations observed during ventilation in CPAP with a B-CPAP system). FIGS. 5, 8, and 11 illustrate embodiments of a flow graph showing the flow delivered 502 during a traditional breath type and the flow delivered 504 by the ventilator during an adjusted breath type (i.e. after the delivered flow has been adjusted to include the normalized flow fluctuation observed during ventilation in CPAP with a B-CPAP system). FIG. 4 illustrates an embodiment of a graph 400 of pressure delivered during a CPAP mode and an adjusted CPAP mode or an M-CPAP mode. FIG. 5 illustrates an embodiment of a graph 500 of flow recorded during a CPAP mode and an adjusted CPAP mode or an M-CPAP mode. FIG. 7 illustrates an embodiment of a graph 700 of pressure recorded during a VC breath type and an adjusted VC breath type. FIG. 8 illustrates an embodiment of a graph 800 of flow delivered during a VC breath type and an adjusted VC breath type. FIG. 10 illustrates an embodiment of a graph 1000 of pressure delivered during a PAV breath type and an adjusted PAV breath type. FIG. 9 illustrates an embodiment of a graph 900 of flow recorded during a PAV breath type and an adjusted PAV breath type.

FIGS. 4, 5, 7, 8, 10, and 11 are exemplary only and are not meant to limit the disclosure. Any known breath type may be adjusted with recorded fluctuations of pressure and/or flow as delivered during a CPAP mode to a different patient with a B-CPAP system. These fluctuations are added to the delivered pressure and/or flow of the breath types to improve patient gas exchange. For example, the added pressure and/or flow fluctuations in the breath type may prevent statistically significant changes in vital signs from baseline measurements, such as a statistically significant decrease in respiration rate (RR), pH, and PaO₂ with a corresponding statistically significant increase in PaCO₂, HCO₃ of the patient.

In some embodiments, the normalized bubble signal 113 is added only to a portion of the breath type control signal 111. For example, the normalized bubble signal 113, which replicates the pressure and flow fluctuations caused by bubbles during CPAP delivery by a ventilator utilizing a B-CPAP system, may only be added to the exhalation portion of the breath type, added to a predetermined number of breaths in the breath type, added to alternating breaths in the breath type, or added to a predetermined period of time in the breath type. For instance, the normalized bubble signal 113 may be applied to alternating periods during weaning.

In some embodiments, the use of the adjusted breath type is determined by the ventilator. The ventilator 100 may determine when to utilize the adjusted breath type based on ventilator parameters. In an alternative embodiment, the use of the adjusted breath type is selected by the operator, clinician, or user. In some embodiments, when only a portion of a breath is adjusted by the normalized bubble signal 113 (e.g., inspiration or exhalation, or a portion of inspiration or exhalation), the ventilator 100 may determine what portion of the breath to adjust. In an alternative embodiment, the operator may select what portion of the breath to adjust with the normalized bubble signal 113.

The ventilator 100 may also include one or more sensors 107 communicatively coupled to ventilator 100. The sensors 107 may be located in the pneumatic system 102, ventilation tubing system 130, and/or on the patient 150. The embodiment of FIG. 1 illustrates a sensor 107 in pneumatic system 102.

Sensors 107 may communicate with various components of ventilator 100, e.g., pneumatic system 102, other sensors 107, expiratory module 108, inspiratory module 104, processor 116, controller 110, trigger module 115, bubble mimic module 117, and any other suitable components and/or modules. In one embodiment, sensors 107 generate output and send this output to pneumatic system 102, other sensors 107, expiratory module 108, inspiratory module 104, processor 116, controller 110, trigger module 115, bubble mimic module 117, and any other suitable components and/or modules.

Sensors 107 may employ any suitable sensory or derivative technique for monitoring one or more patient parameters or ventilator parameters associated with the ventilation of a patient 150. Sensors 107 may detect changes in patient parameters indicative of patient inspiratory triggering or expiratory cycling, for example. Sensors 107 may be placed in any suitable location, e.g., within the ventilatory circuitry or other devices communicatively coupled to the ventilator 100. Further, sensors 107 may be placed in any suitable internal location, such as, within the ventilatory circuitry or within components or modules of ventilator 100. For example, sensors 107 may be coupled to the inspiratory and/or expiratory modules 104, 108 for detecting changes in, for example, circuit pressure and/or flow. In other examples, sensors 107 may be affixed to the ventilatory tubing or may be embedded in the tubing itself. According to some embodiments, sensors 107 may be provided at or near the lungs (or diaphragm) for detecting a pressure in the lungs. Additionally or alternatively, sensors 107 may be affixed or embedded in or near wye-fitting 170 and/or patient interface 180. Any sensory device useful for monitoring changes in measurable parameters during ventilatory treatment may be employed in accordance with embodiments described herein.

For example, in some embodiments, the one or more sensors 107 of the ventilator 100 include an inspiratory flow sensor and/or an expiratory flow sensor. The inspiratory flow sensor may be located in any suitable position for monitoring inspiratory flow, such as at the patient interface 180 or within the ventilation tubing system 130. The inspiratory flow sensor may be monitored by any suitable ventilator component, such as a pressure generating system 102. In one embodiment, the expiratory flow sensor is located in the expiratory limb 134 and is monitored by the expiratory module 108. However, the expiratory flow sensor may be located in any suitable position for monitoring expiratory flow and may be monitored by any suitable ventilator component, such as a pressure generating system 102.

In some embodiments, the ventilator 100 monitors the flow and pressure data gathered by a sensor 107 to determine if a flow and/or pressure threshold has been surpassed. If a threshold has been exceeded, the ventilator 100 may issue an alarm to notify the patient and/or clinician of this threshold breach. The alarm may include any suitable type of notification, such as a visual, an audio, or a vibrational cue. For example, an alarm may create a sound, issue a prompt on the display, flash a light, and/or vibrate to notify the clinician or patient of the exceeded threshold. As discussed above, ventilators that utilized a B-CPAP system have difficulty monitoring flow and/or pressure unlike the ventilator 100 that utilizes the V-CPAP system. Therefore, alarms based on flow and pressure monitoring are difficult in ventilators that utilize the B-CPAP system. The oscillatory nature of the pressure and flow signals may cause problems in monitoring flow and pressure for a ventilator utilizing B-CPAP system. The M-CPAP system processes can process the signals to remove or filter out the oscillatory component since the ventilator has knowledge of the frequency and amplitude that has been injected into the signal. These oscillations also cause difficulty in the B-CPAP system with triggering, spirometry, and cycling events.

Further, the monitoring of flow and/or pressure allows the ventilator 100 to determine and/or monitor the amount of breathing gas that leaks from ventilator 100 (e.g., from the patient circuit, patient interface, valves, etc.) and is not delivered to the patient 150. In some embodiments, gas leakage is determined by the ventilator 100 by monitoring the flow delivered to the patient 150 and by monitoring the flow returned from the patient 150. For example, while the flow returned from the patient 150 will be oscillatory due to the replication of the bubbles, the returned flow can still be averaged or filtered out as discussed above and the result subtracted from the delivered flow in order to quantify the breathing gas leakage. The quantification of leakage may facilitate the proper placement of the patient interface 180. For example, too little leakage may indicate excessive contact between the patient interface 180 and the patient 150 leading to tissue damage, while too much leakage could indicate that the patient is no longer receiving adequate flow. As discussed above, ventilators that utilized B-CPAP systems have difficulty monitoring flow and/or pressure therefore, these ventilators have difficulty monitoring leakage.

As should be appreciated, with reference to the Equation of Motion, ventilatory parameters are highly interrelated and, according to embodiments, may be either directly or indirectly monitored. That is, parameters may be directly monitored by one or more sensors 107, as described above, or may be indirectly monitored or estimated by derivation according to the Equation of Motion or other known relationships.

The pneumatic system 102 may include a variety of other components, including mixing modules, valves, tubing, accumulators, filters, etc.

In one embodiment, the operator interface 120 of the ventilator 100 includes a display 122 communicatively coupled to ventilator 100. Display 122 provides various input screens, for receiving clinician input, and various display screens, for presenting useful information to the clinician. In one embodiment, the display 122 is configured to include a graphical user interface (GUI). The GUI may be an interactive display, e.g., a touch-sensitive screen or otherwise, and may provide various windows and elements for receiving input and interface command operations. Alternatively, other suitable means of communication with the ventilator 100 may be provided, for instance by a wheel, keyboard, mouse, or other suitable interactive device. Thus, operator interface 120 may accept commands and input through display 122.

Display 122 may also provide useful information in the form of various ventilatory data regarding the physical condition of a patient 150. The useful information may be derived by the ventilator 100, based on data collected by a processor 116, and the useful information may be displayed to the clinician in the form of graphs, wave representations, pie graphs, text, or other suitable forms of graphic display. For example, patient data may be displayed on the GUI and/or display 122. Additionally or alternatively, patient data may be communicated to a remote monitoring system coupled via any suitable means to the ventilator 100. In some embodiments, the display 122 may illustrate a normalized bubble signal, the use of a normalized bubble signal, the M-CPAP mode, the adjusted breath type, flow and/or pressure graphs of an adjusted breath type, such as M-CPAP or a previously recorded B-CPAP mode, and/or any other information known, received, or stored by the ventilator 100.

Controller 110 is operatively coupled with pneumatic system 102, signal measurement and acquisition systems, and an operator interface 120 that may enable an operator to interact with the ventilator 100 (e.g., change ventilator settings, select operational modes, view monitored parameters, etc.).

In some embodiments, controller 110 includes memory 112, one or more processors 116, storage 114, and/or other components of the type commonly found in command and control computing devices. Controller 110 may further include a trigger module 115, and bubble mimic module 117, as illustrated in FIG. 1. In alternative embodiments, the trigger module 115, and bubble mimic module 117 are located in other components of the ventilator 100, such as in the pressure generating system 102 (also known as the pneumatic system 102).

The memory 112 includes non-transitory, computer-readable storage media that stores software that is executed by the processor 116 and which controls the operation of the ventilator 100. In an embodiment, the memory 112 includes one or more solid-state storage devices such as flash memory chips. In an alternative embodiment, the memory 112 may be mass storage connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

Ventilators 100, depending on their mode of operation, may trigger automatically and/or in response to a detected change in a ventilator parameter. Trigger detection is necessary in order to deliver an spontaneous mode of ventilation (a mode that requires the patient to initiate inspiration). The trigger module 115 receives and/or determines one or more inspiration trigger thresholds. In some embodiments, the trigger module 115 receives an inspiration trigger threshold from operator input. In other embodiments, the trigger module 115 determines an inspiration trigger threshold based on ventilator and/or patient parameters. During exhalation, in one embodiment, the trigger module 115 monitors ventilator and/or patient parameters and compares these parameters to one or more inspiration trigger thresholds to determine if the parameters meet and/or exceed the inspiration trigger thresholds. If the trigger threshold is exceeded, the trigger module 115 commands the ventilator 100 to trigger inspiration. If the trigger threshold is not exceeded, the trigger module 115 does not command the ventilator 100 to perform any function. However, in some embodiments after a predetermined amount of time has passed without a patient trigger, the trigger module 115 commands the ventilator 100 to trigger ventilation to prevent insufficient ventilation of the patient 150.

Specifically, the ventilator may detect patient effort via a pressure-monitoring method, a flow-monitoring method, direct or indirect measurement of nerve impulses, or any other suitable method. Sensing devices may be either internal or distributed and may include any suitable sensing device, as described further herein. In addition, the sensitivity of the ventilator to changes in pressure and/or flow may be adjusted such that the ventilator may properly detect the patient effort, i.e., the lower the pressure or flow change setting the more sensitive the ventilator may be to patient triggering. As discussed above, ventilators that deliver CPAP by utilizing a B-CPAP system have difficulty monitoring flow and/or pressure; and therefore are not able to determine a patient trigger. Therefore, ventilators that utilize a B-CPAP system could not deliver spontaneous mode breath types, such as PAV. The ventilator 100 is capable of monitoring flow and/or pressure and therefore, can deliver breath types in response to a detected trigger.

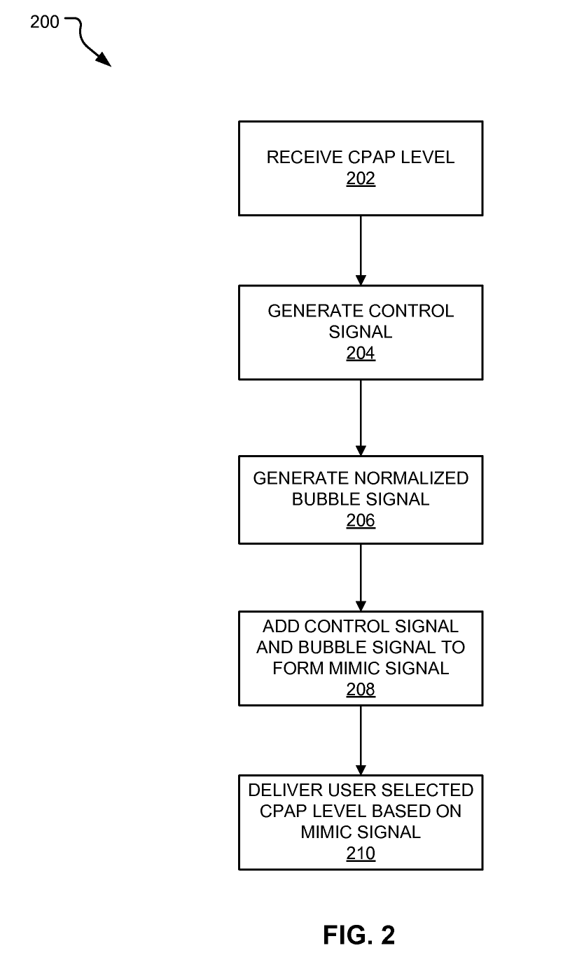

FIG. 2 illustrates an embodiment of a method 200 for ventilating a patient according to a M-CPAP mode on a ventilator. The M-CPAP mode delivers fluctuations in flow and/or pressure to the patient that mimic pressure and/or flow fluctuations delivered by a different ventilator during a CPAP mode utilizing a B-CPAP system. These fluctuations are added to the delivered pressure and/or flow of the breath types to improve patient gas exchange. For example, the added pressure and/or flow fluctuations in the breath type may prevent statistically significant changes in vital signs from baseline measurements, such as a statistically significant decrease in respiration rate (RR), pH, and PaO₂ with a corresponding statistically significant increase in PaCO₂, HCO₃ of the patient.

As illustrated, method 200 includes receiving operation 202. During the receiving operation 202, the ventilator receives a CPAP level. The received CPAP level is input or selected by an operator. The CPAP level may be any suitable level of pressure for delivery during a CPAP mode, such as 3 cm of H₂O.

Further, method 200 includes a generating control signal operation 204. During the generating control signal operation 204, the ventilator generates a control signal for controlling breathing gas delivery and an exhalation valve to obtain the received CPAP level. As discussed above the exhalation valve and/or flow delivery can affect the amount of pressure and/or flow delivered to the patient by the ventilator. In some embodiments, the control signal is determined by a controller. In other embodiments, the control signal is determined by the pressure generating system of the ventilator. In some embodiments, a gain may be added to the control signal during control signal operation 204 to adjust the amplitude of the oscillations.

Additionally, method 200 includes a generating normalized bubble signal operation 206. During the generating normalized bubble signal operation 206, the ventilator generates a normalized bubble signal. In one embodiment, the normalized bubble signal is normalization of a signal based on recorded flow fluctuations and/or recorded pressure fluctuations during ventilation of a prior patient utilizing a B-CPAP system at a predetermined CPAP level for a set time period which is looped. The normalized bubble signal determines how much the pressure and/or flow fluctuates from the intended delivered pressure and/or flow and then utilizes just these fluctuations above and below the intended flow and/or pressure delivery to determine the normalized signal. FIG. 9 illustrates an embodiment of a graph 900 of normalized pressure fluctuations based on pressure fluctuation observed in graph 600 of FIG. 6.

In an alternative embodiment, the normalized bubble signal is a normalized signal based on theoretical flow fluctuations and/or theoretical pressure fluctuations caused by mathematically estimated bubbles during ventilation of a patient utilizing a B-CPAP system at a predetermined CPAP level for a set time period which is randomized. Mathematical equations are known that mathematically estimate bubbles and the displacement of flow and/or pressure caused by these estimated bubbles in a predetermined water column. These equations may be utilized to estimate the fluctuation caused by bubbles in a water column of a B-CPAP system at different CPAP settings. These estimations may be utilized to fluctuate the flow and/or pressure delivered during the delivery of CPAP with a V-CPAP system. However, bubbles produced in the water column will appear randomly and create random fluctuations in the delivered pressure and flow during ventilation of a patient with a ventilator utilizing a B-CPAP system. Accordingly, the theoretically calculated pressure and/or flow oscillations may be randomized to account for the randomness of the B-CPAP delivery system. Similar to the normalized bubble signal discussed above, the theoretically calculated fluctuations may be determined based on a set CPAP setting for a predetermined amount of time and then looped.

Next method 200 includes an adding operation 208. The ventilator during adding operation 208 adds the control signal and the normalized bubble signal to form a mimic signal. Similar to the control signal, the mimic signal is instructions for controlling breath delivery and/or the exhalation valve to deliver the selected breath type with the mimicked pressure and/or flow variations recorded from bubbles during ventilation of a prior patient by a ventilator with B-CPAP system. In some embodiments, the adding operation is performed by a controller. In an alternative embodiment, the adding operation is performed by a pressure generating system. In some embodiments, the adding operation is performed by a bubble mimic module.

Method 200 includes a delivering operation 210. The ventilator during delivering operation 210 delivers the selected CPAP level to the patient with fluctuations in flow and/or pressure based on the mimic signal. For example, FIGS. 4 and 5 illustrate embodiments of a delivered pressure 404 and a delivered flow 504 with the fluctuation in flow and pressure based on the mimic signal. FIG. 4 illustrates an embodiment of a graph 400 of pressure delivered during a CPAP mode and an adjusted CPAP mode or an M-CPAP mode. FIG. 5 illustrates an embodiment of a graph 500 of flow recorded during a CPAP mode and an adjusted CPAP mode or an M-CPAP mode.

In other embodiments, method 200 includes a display operation. The ventilator during the display operation displays any suitable information for display on a ventilator. In one embodiment, the display operation displays at least one of a normalized bubble signal, the use of a normalized bubble signal, the M-CPAP mode, the adjusted breath type, flow and/or pressure graphs of an adjusted breath type, such as M-CPAP, and/or any other information known, received, or stored by the ventilator.

In some embodiments, a microprocessor-based ventilator that accesses a computer-readable medium having computer-executable instructions for performing the method of ventilating a patient with a medical ventilator is disclosed. This method includes repeatedly performing the steps disclosed in method 200 above and/or as illustrated in FIG. 2.

In some embodiments, the ventilator system includes: means for receiving a user selected CPAP level; means for generating a control signal for controlling breathing gas delivery and an exhalation valve to obtain the user selected CPAP level; means for generating a normalized bubble signal; means for adding the control signal and the normalized bubble signal to form a mimic signal; and means for delivering the user selected CPAP level to the patient with fluctuations in at least one of flow and pressure based on the mimic signal.

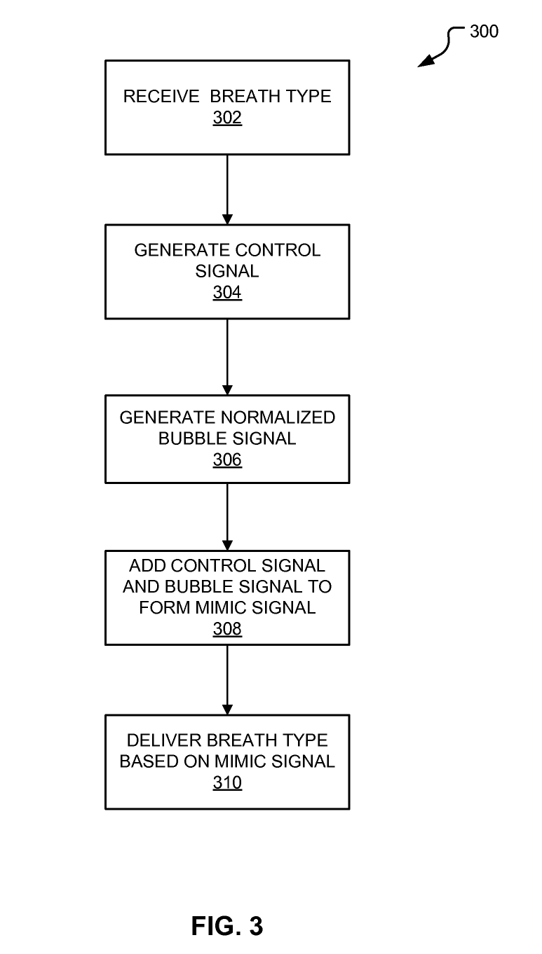

FIG. 3 illustrates a method 300 for delivering an adjusted breath type during ventilation of a patient on a ventilator. The adjusted breath type delivers fluctuations in flow and/or pressure to the patient that mimic pressure and/or flow fluctuations delivered by a different ventilator during a CPAP mode utilizing a B-CPAP system. These fluctuations are added to the delivered pressure and/or flow of the breath types to improve patient gas exchange. For example, the added pressure and/or flow fluctuations in the breath type may prevent statistically significant changes in vital signs from baseline measurements, such as a statistically significant decrease in respiration rate (RR), pH, and PaO₂ with a corresponding statistically significant increase in PaCO₂, HCO₃ of the patient.

As illustrated, method 300 includes a receiving operation 302. During the receiving operation 302, the ventilator receives a breath type. In some embodiments, the received breath type is determined by the ventilator. The ventilator may determine the breath type based on ventilator parameters. In other embodiments, the received breath type is input or selected by an operator. The breath type may include VC, VS, PC, PS VC+, PAV, CPAP and/or any other known and utilized breath type for ventilation of a patient.

Further, method 300 includes a generating control signal operation 304. During the generating control signal operation 304, the ventilator generates a control signal for controlling breathing gas delivery and an exhalation valve to implement the received breath type. As discussed above the exhalation valve and/or flow delivery can affect the amount of pressure and/or flow delivered to the patient by the ventilator. In some embodiments, the control signal is determined by a controller. In other embodiments, the control signal is determined by the pressure generating system of the ventilator.

Additionally, method 300 includes a generating normalized bubble signal operation 306. During the generating normalized bubble signal operation 306, the ventilator generates a normalized bubble signal. The generating normalized bubble signal operation 306 is similar to the generating normalized bubble signal operation 206 described above in method 200. In one embodiment, the normalized bubble signal is normalized of a signal based on recorded flow fluctuations and/or recorded pressure fluctuations during ventilation of a prior patient utilizing a B-CPAP system at a predetermined CPAP level for a set time period which is looped. The normalization bubble signal determines how much the pressure and/or flow fluctuates from the intended delivered pressure and/or flow and then utilizes just these fluctuations above and below the intended flow and/or pressure delivery to determine the normalized signal. In some embodiments, a gain may be added to the control signal during control signal operation 204 to adjust the amplitude of the oscillations. FIG. 9 illustrates an embodiment of a graph 900 of normalized pressure fluctuations based on pressure fluctuation observed in graph 600 of FIG. 6.

In an alternative embodiment, the normalized bubble signal is a normalized signal based on theoretical flow fluctuations and/or theoretical pressure fluctuations caused by mathematically estimated bubbles during ventilation of a patient utilizing a B-CPAP system at a predetermined CPAP level for a set time period which is randomized. Mathematical equations are known that mathematically estimate bubbles and the displacement of flow and/or pressure caused by these estimated bubbles in a predetermined water column. These equations may be utilized to estimate the fluctuation caused by bubbles in a water column of a B-CPAP system at different CPAP settings. These estimations may be utilized to fluctuate the flow and/or pressure delivered during the delivery of CPAP with a V-CPAP system. However, bubbles produced in the water column will appear randomly and create random fluctuations in the delivered pressure and flow during ventilation of a patient with a ventilator utilizing a B-CPAP system. Accordingly, the theoretically calculated pressure and/or flow oscillations may be randomized to account for the randomness of the B-CPAP delivery system. Similar to the normalized bubble signal discussed above, the theoretically calculated fluctuations may be determined based on a set CPAP setting for a predetermined amount of time and then looped.

Next method 300 includes an adding operation 308. The ventilator during adding operation 308 adds the control signal for the received breath type and the normalized bubble signal to form a mimic signal. The adding operation 308 is similar to the adding operation 208 described above in method 200. Similar to the control signal, the mimic signal is instructions for controlling breath delivery and/or the exhalation valve to deliver the selected breath type with the mimicked pressure and/or flow variations recorded from bubbles during ventilation of a different patient by a ventilator with B-CPAP system. In some embodiments, the adding operation is performed by a controller. In an alternative embodiment, the adding operation is performed by a pressure generating system. In some embodiments, the adding operation is performed by a bubble mimic module.

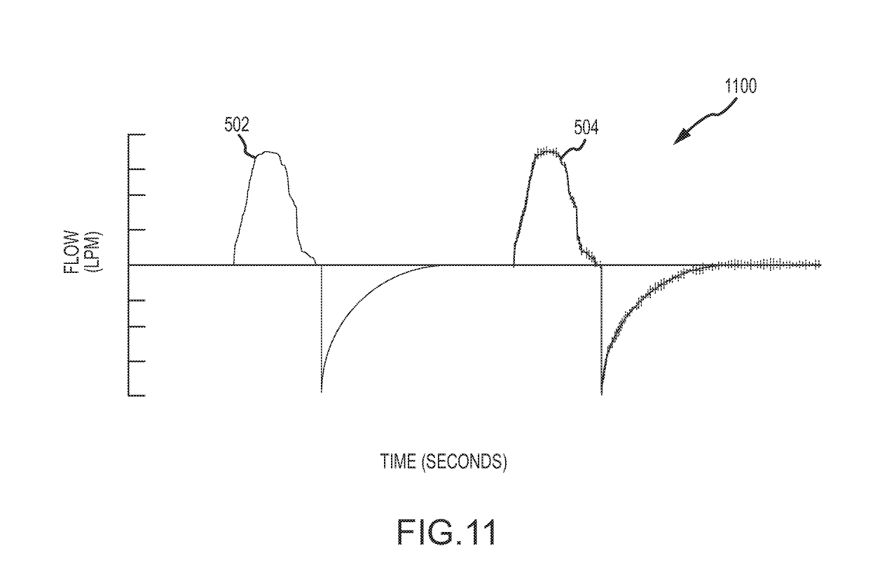

Method 300 includes a delivering operation 310. The ventilator during delivering operation 310 delivers the received breath type to the patient with the fluctuation in flow and/or pressure based on the mimic signal. For example, FIGS. 4 and 5 illustrate embodiments of a delivered pressure 404 and a delivered flow 504 with the fluctuation in flow and pressure based on the mimic signal for a CPAP mode. FIG. 4 illustrates an embodiment of a graph 400 of pressure delivered during a CPAP mode and an adjusted CPAP mode or an M-CPAP mode. FIG. 5 illustrates an embodiment of a graph 500 of flow recorded during a CPAP mode and an adjusted CPAP mode or an M-CPAP mode. In an additional example, FIGS. 7 and 8 illustrate embodiments of a recorded pressure 404 and a delivered flow 504 with the fluctuation in flow and pressure based on the mimic signal for a VC breath type. FIG. 7 illustrates an embodiment of a graph 700 of pressure delivered during a VC breath type and an adjusted VC breath type. FIG. 8 illustrates an embodiment of a graph 800 of flow delivered during a VC breath type and an adjusted VC breath type. In further embodiments, FIGS. 10 and 11 illustrate embodiments of a delivered pressure 404 and a recorded flow 504 with the fluctuation in flow and pressure based on the mimic signal for a PAV breath type. FIG. 10 illustrates an embodiment of a graph 1000 of pressure recorded during a PAV breath type and an adjusted PAV breath type. FIG. 11 illustrates an embodiment of a graph 1100 of flow delivered during a PAV breath type and an adjusted PAV breath type

In some embodiments, method 300 includes a display operation. The ventilator during the display operation, similar to the display operation discussed for method 200, displays any suitable information for display on a ventilator. In one embodiment, the display operation displays at least one of a normalized bubble signal, the use of a normalized bubble signal, the M-CPAP mode, the adjusted breath type, flow and/or pressure graphs of an adjusted breath type, and/or any other information known, received, or stored by the ventilator.

In some embodiments, a microprocessor-based ventilator that accesses a computer-readable medium having computer-executable instructions for performing the method of ventilating a patient with a medical ventilator is disclosed. This method includes repeatedly performing the steps disclosed in method 300 above and/or as illustrated in FIG. 3.

In some embodiments, the ventilator system includes: means for receiving a breath type; means for generating a control signal for operating at least one of an exhalation valve and a pressure generating system to provide the breath type; means for generating a normalized bubble signal for operating the at least one of the exhalation valve and the pressure generating system; means for adding the control signal and the normalized bubble signal to form a mimic signal; means for sending the mimic signal to the at least one of the exhalation valve and the pressure generating system; and means for delivering the breath type to the patient with fluctuations in at least one of flow and pressure based on the mimic signal.

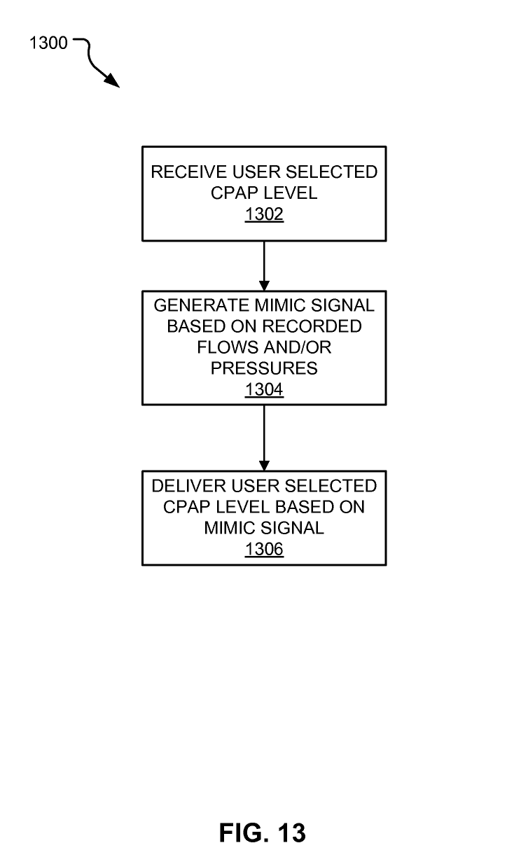

FIG. 13 illustrates an embodiment of a method 1300 for ventilating a patient according to a M-CPAP mode on a ventilator. The M-CPAP mode delivers fluctuations in flow and/or pressure to the patient based on recorded pressure and/or flow fluctuations delivered by a different ventilator during a CPAP mode utilizing a B-CPAP system. These fluctuations are added to the delivered pressure and/or flow of the breath types to improve patient gas exchange. For example, the pressure and/or flow fluctuations in the delivered CPAP mode may prevent statistically significant changes in vital signs from baseline measurements, such as a statistically significant decrease in respiration rate (RR), pH, and PaO₂ with a corresponding statistically significant increase in PaCO₂, HCO₃ of the patient.

As illustrated, method 1300 includes a receiving operation 1302. During the receiving operation 1302, the ventilator receives a CPAP level. The receiving operation 1302 is similar to the receiving operation 202 described above for method 200. The received CPAP level is selected or input by an operator.

Additionally, method 1300 includes a generating mimic signal operation 1304. During the generating mimic signal operation 1304, the ventilator generates a mimic signal based on previously recorded flow fluctuations and/or recorded pressure fluctuations during ventilation of a prior patient utilizing a B-CPAP system at a predetermined CPAP level for a set time period which is looped. FIG. 6 illustrates an embodiment of a graph 600 of recorded delivered pressure fluctuations 403 during ventilation of a prior patient by a ventilator utilizing a B-CPAP system. FIG. 12 illustrates an embodiment of a graph 1200 of recorded delivered flow fluctuations 405 during ventilation of a patient by a ventilator utilizing a B-CPAP system. The generated mimic signal is designed to mimic the pressure and/or flow fluctuations as was previously delivered to the prior patient for a set CPAP level for a set time period by controlling the expiratory module and/or the gas delivery of the V-CPAP system. The mimic signal, in this embodiment, loops the mimic signal to deliver fluctuations past the set time period of the recorded signal. In some embodiments, the ventilator stores numerous different recorded flow fluctuations and/or recorded pressure fluctuations during ventilation of the same prior patient utilizing a B-CPAP system at any desired number of different CPAP level settings for the same set time period. The storing of numerous recorded signals provides for numerous mimic signals, which allow the ventilator to deliver substantially similar flow and/or pressure fluctuations as delivered by the ventilator with a B-CPAP system at the same set CPAP setting.

Method 1300 includes a delivering operation 1306. The ventilator during delivering operation 1306 delivers the received CPAP level to the patient with substantially similar fluctuations in flow and/or pressure delivered to a prior patient during a CPAP mode at the received CPAP level by a ventilator with B-CPAP system based on the mimic signal. For example, FIGS. 4 and 5 illustrate embodiments of a delivered pressure 404 and a delivered flow 504 with the fluctuation in flow and pressure based on the mimic signal for a CPAP mode. For example, if the received CPAP level is 5 cm H₂O, then the mimic signal is generated based on a recorded delivery of 5 cm H₂O to a prior patient by a ventilator utilizing a B-CPAP system. The delivery of the CPAP level by the V-CPAP system is then based on this generated mimic signal to deliver substantially similar fluctuations in pressure and/or flow as recorded from the patient utilizing the B-CPAP system at the same CPAP level (5 cm H₂O).

In some embodiments, method 1300 includes a display operation. The ventilator during the display operation, similar to the display operation discussed for method 200, displays any suitable information for display on a ventilator. In one embodiment, the display operation displays at least one of the M-CPAP mode, a previously recorded flow and/or pressure delivery by a ventilator during CPAP with a B-CPAP system, flow and/or pressure graphs of a delivered M-CPAP and/or any other information known, received, or stored by the ventilator.

In some embodiments, a microprocessor-based ventilator that accesses a computer-readable medium having computer-executable instructions for performing the method of ventilating a patient with a medical ventilator is disclosed. This method includes repeatedly performing the steps disclosed in method 1300 above and/or as illustrated in FIG. 13.

In other embodiments, the ventilator system includes means for receiving a user selected CPAP level; means for generating a mimic signal based on recorded pressure and/or flows from patients ventilated with a B-CPAP system; and means for delivering the user selected CPAP level based on the generated mimic signal.

In some embodiments, methods 200, 300, or 1300 may further include a monitoring operation. The ventilator during the monitor operation monitors flow and/or pressure. The flow and/or pressure may be monitored by any suitable ventilator sensor. In additional embodiments, methods 200, 300, or 1300 may further include an execution operation. During the execution operation the ventilator executes an alarm if the monitored flow and/or pressure exceed a predetermined threshold. The predetermined threshold may be determined by the ventilator or selected by a clinician. If the monitored flow and/or pressure does not exceed the flow and/or pressure predetermined threshold, the ventilator does not execute an alarm. The alarm may be any suitable alarm for a ventilation system. In further embodiments where flow and/or pressure are monitored by the ventilator, methods 200, 300, or 1300 may further include a leakage operation. During the leakage operation, the ventilator may monitor the amount of breathing gas leaked from the ventilator. If the gas leakage is too high or too low, the ventilator during leakage operation may further execute an alarm.

In other embodiments, methods 200, 300, or 1300 may further include a mode switch operation. The ventilator during the mode switch operation switches from a mandatory mode to a spontaneous mode. In some embodiments, the ventilator may switch to a spontaneous mode based on monitored ventilator parameters. In other embodiments, the ventilator switches to a spontaneous mode based on user selection.

Those skilled in the art will recognize that the methods and systems of the present disclosure may be implemented in many manners and as such are not to be limited by the foregoing exemplary embodiments and examples. In other words, functional elements being performed by a single or multiple components, in various combinations of hardware and software or firmware, and individual functions, can be distributed among software applications at either the client or server level or both. In this regard, any number of the features of the different embodiments described herein may be combined into single or multiple embodiments, and alternate embodiments having fewer than or more than all of the features herein described are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter.

Numerous other changes may be made which will readily suggest themselves to those skilled in the art and which are encompassed in the spirit of the disclosure and as defined in the appended claims. While various embodiments have been described for purposes of this disclosure, various changes and modifications may be made which are well within the scope of the present invention. Numerous other changes may be made which will readily suggest themselves to those skilled in the art and which are encompassed in the spirit of the disclosure and as defined in the claims.

What is claimed is:

1. A method for ventilating a patient with a ventilator, comprising:
receiving a user selected Continuous Positive Airway Pressure (CPAP) level;
generating a control signal for controlling breathing gas delivery and an exhalation valve to implement the user selected CPAP level;
generating a normalized bubble signal;
adding the control signal and the normalized bubble signal to form a mimic signal; and
delivering the user selected CPAP level to the patient with fluctuations in at least one of flow and pressure based on the mimic signal.

2. The method of claim 1, further comprising monitoring at least one of the flow and the pressure during ventilation.

3. The method of claim 2, further comprising executing an alarm when at least one of the monitored flow and the monitored pressure exceed a predetermined threshold.

4. The method of claim 2, further comprising monitoring leakage based on the step of monitoring flow and the monitored pressure.

5. The method of claim 2, further comprising:
switching to a spontaneous mode from a mandatory.

6. The method of claim 1, wherein the normalized bubble signal is based on at least one of recorded flow fluctuations and recorded pressure fluctuations during ventilation of a prior patient utilizing a bubble Continuous Positive Airway Pressure (B-CPAP) system at a predetermined CPAP level for a set time period which is looped.

7. The method of claim 1, wherein the normalized bubble signal is based on at least one of theoretical flow fluctuations and theoretical pressure fluctuations caused by mathematically estimated bubbles for set time period which is randomized.

8. The method of claim 1, wherein gas exchange exhibited by the patient is similar to the gas exchange exhibited by a different patient ventilated with a bubble Continuous Positive Airway Pressure (B-CPAP) system.

9. The method of claim 1, wherein the patient exhibits at least one of a decrease in respiration rate, a decrease in pH, a decrease in PaO₂, an increase in PaCO₂, and an increase in HCO₃ compared to a different patient ventilated with a ventilator-derived Continuous Positive Airway Pressure (V-CPAP) system that does not utilize a mimic signal.

10. A method for ventilating a patient with a ventilator, comprising:
receiving a breath type;
generating a control signal for controlling breathing gas delivery and an exhalation valve to implement the breath type;
generating a normalized bubble signal;
adding the control signal and the normalized bubble signal to form a mimic signal; and
delivering the breath type to the patient with fluctuations in at least one of flow and pressure based on the mimic signal.

11. The method of claim 10, wherein the normalized bubble signal is based on at least one of recorded flow fluctuations and recorded pressure fluctuations during ventilation of a prior patient utilizing a bubble Continuous Positive Airway Pressure (B-CPAP) system at a predetermined Continuous Positive Airway Pressure (CPAP) level for a set time period which is looped.

12. The method of claim 10, wherein the normalized bubble signal is based on at least one of theoretical flow fluctuations and theoretical pressure fluctuations caused by mathematically estimated bubbles for set time period which is randomized.

13. The method of claim 10, wherein the patient exhibits at least one of a decrease in respiration rate, a decrease in pH, a decrease in PaO₂, an increase in PaCO₂, and an increase in HCO₃ compared to a different patient ventilated with a ventilator-derived Continuous Positive Airway Pressure (V-CPAP) system that does not utilize a mimic signal.

14. The method of claim 10, wherein the breath type is at least one of pressure support, pressure control, pressure assist, volume control, volume-targeted-pressure-control, and volume support breath types.

15. The method of claim 10, wherein the breath type is at least one of a mandatory mode, a spontaneous mode, and a weaning mode.

16. A ventilator system comprising:
a pressure generating system adapted to generate a flow of breathing gas;
the pressure generating system includes an exhalation valve, the exhalation valve controls release of respiratory gases from a patient to the atmosphere;
a ventilation tubing system including a patient interface for connecting the pressure generating system to the patient; and
a bubble mimic module, the bubble mimic module generates a normalized bubble signal and adds the normalized bubble signal to a control signal for a breath type to form a mimic signal,
wherein the pressure generating system delivers the breath type to the patient with fluctuations in at least one of flow and pressure based on the mimic signal from the bubble mimic module.

17. The ventilator system of claim 16, further comprising:
at least one sensor operatively coupled to at least one of the pressure generating system, the patient, and the ventilation tubing system, wherein the at least one sensor is capable of monitoring flow,
wherein the monitored flow is utilized to determine at least one of an alarm based on a predetermined threshold and an amount of breathing gas leaking from the ventilator system.

18. The ventilator system of claim 17, further comprising a controller, wherein the controller filters out the delivered flow oscillations from the monitor flow based on data received from the mimic module.

19. The ventilator system of claim 16, further comprising:
a display in communication with at least one of the pressure generating system, the display illustrates at least one of the normalized bubble signal, a use of the normalized bubble signal, a mimicked-bubble Continuous Positive Airway Pressure (M-CPAP) mode, an adjusted breath type, a delivered pressure graph, and a delivered flow graph.

20. The ventilator system of claim 16, wherein the breath type is at least one of a pressure support, pressure control, pressure assist, volume control, volume-targeted-pressure-control, and volume support.

21. The ventilator system of claim 16, wherein the normalized bubble signal is based on at least one of recorded flow fluctuations and recorded pressure fluctuations during ventilation of a prior patient utilizing a bubble Continuous Positive Airway Pressure (B-CPAP) system at a predetermined Continuous Positive Airway Pressure (CPAP) level for a set time period which is looped.

22. The ventilator system of claim 16, further comprising:
at least one sensor operatively coupled to at least one of the pressure generating system, the patient, and the ventilation tubing system, wherein the at least one sensor is capable of monitoring pressure,
wherein the monitored pressure is utilized to determine at least one of an alarm based on a predetermined threshold and an amount of breathing gas leaking from the ventilator system.

23. The ventilator system of claim 22, further comprising a controller, wherein the controller filters out the delivered pressure oscillations from the monitor pressure based on data received from the mimic module.

24. A computer-readable medium having computer-executable instructions for performing a method of ventilating a patient with a ventilator, the method comprising:
receiving a breath type;
repeatedly generating a control signal for operating at least one of an exhalation valve and a pressure generating system to provide the breath type;
generating a normalized bubble signal for operating the at least one of the exhalation valve and the pressure generating system;
repeatedly adding the control signal and the normalized bubble signal to form a mimic signal;
repeatedly sending the mimic signal to the at least one of the exhalation valve and the pressure generating system; and
repeatedly delivering the breath type to the patient with fluctuations in at least one of flow and pressure based on the mimic signal.

25. A ventilator system, comprising:
means for receiving a breath type;
means for generating a control signal for operating at least one of an exhalation valve and a pressure generating system to provide the breath type;
means for generating a normalized bubble signal for operating the at least one of the exhalation valve and the pressure generating system;
means for adding the control signal and the normalized bubble signal to form a mimic signal;
means for sending the mimic signal to the at least one of the exhalation valve and the pressure generating system; and
means for delivering the breath type to a patient with fluctuations in at least one of flow and pressure based on the mimic signal.
